(19) United States
(12) Patent Application Publication
(10) Pub. No.: US 2009/0153035 A1
(43) Pub. Date: Jun. 18, 2009

(54) Heteroaromatic cycle-containing compound, method of preparing the same and organic light emitting device comprising the same

(76) Inventors: Jung-Han Shin, Suwon-si (KR); Seung-Gak Yang, Suwon-si (KR); Hee-Yeon Kim, Suwon-si (KR); Chang-Ho Lee, Suwon-si (KR); Hee-Joo Ko, Suwon-si (KR)

Correspondence Address: ROBERT E. BUSHNELL & LAW FIRM, 2029 K STREET NW, SUITE 600, WASHINGTON, DC, 20006-1004, US

(21) Appl. No.: 12/213,097

(22) Filed: Jun. 13, 2008

(30) Foreign Application Priority Data
Jun. 15, 2007 (KR) 10-2007-0059101

(51) Int. Cl. H01J 1/63; C07D 471/04; C07D 487/04

(52) U.S. Cl. 313/504; 546/121; 544/281

(57) ABSTRACT

Provided are a heteroaromatic cycle-containing compound used for an organic light emitting diode, represented by Formula 1:

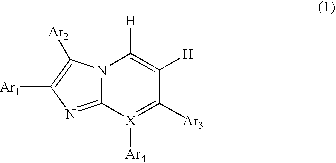

wherein X is N or C; Ar₁ is a phenyl group, a C₆-C₂₀ aryl group, a C₇-C₂₀ arylalkyl group, a C₇-C₂₀ arylalkoxy group, a C₆-C₂₀ arylamino group, a C₆-C₂₀ heteroarylamino group, or a C₂-C₂₀ hetero ring group; and Ar₂, Ar₃ and Ar₄ are each independently hydrogen, a cyano group, a hydroxyl group, a nitro group, halogen, phenyl, a C₁-C₂₀ alkyl group, a C₁-C₂₀ alkoxy group, a C₆-C₂₀ aryl group, a C₇-C₂₀ arylalkyl group, a C₂-C₂₀ alkylalkoxy group, a C₇-C₂₀ arylalkoxy group, a C₆-C₂₀ arylamino group, a C₁-C₂₀ alkylamino group, a C₆-C₂₀ heteroarylamino group, or a C₂-C₂₀ hetero ring group, wherein, when X is N, Ar₄ is a lone electron pair, and when X is C, Ar₃ and Ar₄ are alternatively bound together to form a carbon ring.

### CROSS-REFERENCE TO RELATED PATENT APPLICATION AND CLAIM OF PRIORITY

This application claims the benefit of Korean Patent Application No. 10-2007-0059101, filed on Jun. 15, 2007, in the Korean Intellectual Property Office, the disclosure of which is incorporated herein in its entirety by reference.

### BACKGROUND OF THE INVENTION

1. Field of the Invention

The present invention relates to a heteroaromatic cycle-containing compound for use in an organic light emitting diode, a method of preparing the same and an organic light emitting diode comprising the heteroaromatic cycle-containing compound.

2. Description of the Related Art

Electroluminescent emitting devices, which are self-emissive display devices, have drawn attention for their advantages which are wide viewing angles, high contrast, and a short response time. Electroluminescent emitting devices are classified into inorganic light emitting devices using an inorganic compound and organic light emitting devices (OLED) using an organic compound. Much research has been conducted on OLEDs because OLEDs have higher luminance, a higher turn-on voltage and a quicker response time than those of inorganic light emitting devices, and can also display multiple color images.

In general, OLEDs can have various structures such as an anode/organic emissive layer/cathode structure, or an anode/organic emissive layer/hole blocking layer/cathode structure, an anode/organic emissive layer/electron transport layer/cathode structure or an anode/organic emissive layer/hole blocking layer/electron injection layer/cathode structure.

Metal complex can be used as a material for transporting electrons, and examples of the metal complex include aluminum(III) tris(8-hydroxyquinolate) (Alq₃), bis(10-hydroxybenzo[h]quinolinato)beryllium (BeBq₂), 2,9-dimethyl-4,7-diphenyl-1,10-phenanthroline (BCP), and the like. Alq₃ has high stability, but its properties need to be improved. In the case of BeBq₂ and BCP, due to stacking of aromatic rings between molecules, electron transportation is excellent. However, it is known that stability of the material is low. Thus, life-time, efficiency and consumption power properties have not reached satisfactory levels. Accordingly, there is still a need for improvement.

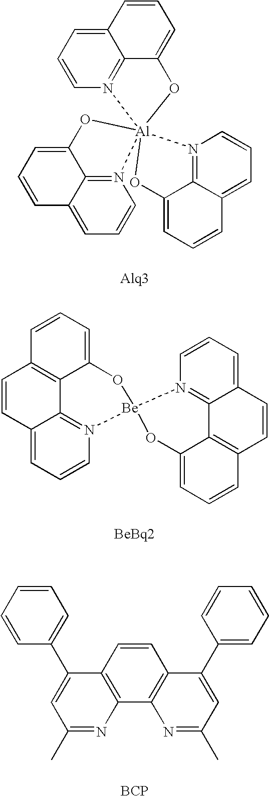

### SUMMARY OF THE INVENTION

The present invention provides a heteroaromatic cycle-containing compound.

The present invention provides an improved organic light emitting diode.

According to an aspect of the present invention, there is provided a heteroaromatic cycle-containing compound for use in an organic light emitting diode, represented by Formula 1 below:

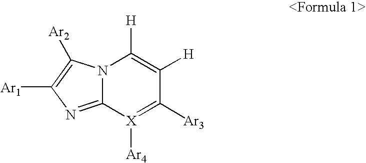

wherein X is N or C;

Ar₁ is a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group; and

Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring.

According to another aspect of the present invention, there is provided a method of preparing the heteroaromatic cycle-containing compound represented by Formula 1 below for use in an organic light emitting diode, comprising: reacting an imidazole derivative (B′) and a boronic acid derivative (C′):

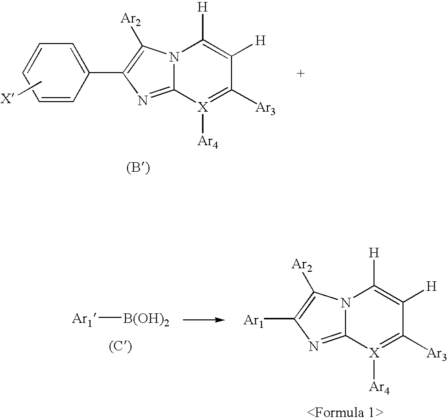

wherein X is N or C;

X′ is a halogen atom;

Ar₁′ and Ar₁ are each independently a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group; and

Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring).

According to another aspect of the present invention, there is provided an organic light emitting diode comprising a single-layered or multi-layered organic layer disposed between a first electrode and a second electrode, wherein the organic layer comprises the heteroaromatic cycle-containing compound represented by Formula 1.

### BRIEF DESCRIPTION OF THE DRAWINGS

The above and other features and advantages of the present invention will become more apparent by describing in detail exemplary embodiments thereof with reference to the attached drawings in which:

FIGS. 1A and 1B are schematic sectional views illustrating structures of a general organic light emitting diode; and

FIG. 2 is a graph showing current-voltage properties of Examples and Comparative Example.

### DETAILED DESCRIPTION OF THE INVENTION

The present invention will now be described more fully with reference to the accompanying drawings, in which exemplary embodiments of the invention are shown.

A heteroaromatic cycle-containing compound used for an organic light emitting diode, according to an embodiment of the present invention has excellent stability and provides good electron transportation, and thus can be effectively used as an organic layer material. Using the compound, an organic light emitting diode that operates at a low voltage and has long life-time can be obtained.

The heteroaromatic cycle-containing compound used for an organic light emitting diode may be represented by Formula 1 below.

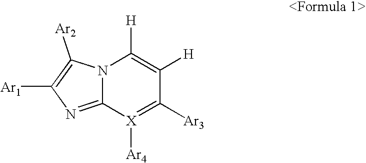

wherein X is N or C;

Ar₁ is a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group; and

Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair (i.e., a lone electron pair of the nitrogen of X), and when X is C, Ar₃ and Ar₄ may be alternatively bound to each other to form a saturated or unsaturated carbon ring. That is, Ar₃ and Ar₄ include the groups formed by being bound to each other as well as the listed groups for Ar₃ and Ar₄.

At least one hydrogen atom of the phenyl group of Formula 1 can be substituted with a halogen atom, a hydroxyl group, a nitro group, a cyano group, an amino group, an amidino group, hydrazine, hydrazone, a carboxyl group or a salt thereof, a sulfonic acid group or a salt thereof, a phosphoric acid or a salt thereof, a C₁-C₂₀ alkyl group, a C₁-C₂₀ alkenyl group, a C₁-C₂₀ alkynyl group, a C₆-C₂₀ aryl group, a C₇-C₂₀ arylalkyl group, a C₂-C₂₀ heteroaryl group, or a C₃-C₂₀ heteroarylalkyl group.

The unsubstituted C₆-C₂₀ aryl group used in Formula 1 is used alone or in combination, and refers to at least one aromatic carbon ring having 6-20 carbon atoms, wherein the rings can be attached by a pendant manner. Examples of the aryl group include phenyl, naphthyl, tetrahydronaphthyl, and the like. At least one hydrogen atom of the aryl group can be substituted with the substituent described above for the phenyl group.

The unsubstituted C₇-C₂₀ arylalkyl group used in Formula 1 refers to a group in which at least one of hydrogen atoms in the aryl group as defined above is substituted with a lower alkyl group, such as methyl, ethyl, propyl, or the like. For example, the arylalkyl group can be benzyl, phenylethyl, and the like. At least one hydrogen atom of the arylalkyl group can be substituted with the substituent described above for the phenyl group.

The unsubstituted C₁-C₂₀ alkyl group used in Formula 1 specifically includes methyl, ethyl, propyl, isobutyl, sec-butyl, pentyl, iso-amyl, hexyl, and the like. At least one hydrogen atom of the alkyl group can be substituted with the substituent described above for the phenyl group.

The unsubstituted C₁-C₂₀ alkoxy group used in Formula 1 specifically includes methoxy, ethoxy, phenyloxy, cyclohexyloxy, naphthyloxy, isopropyloxy, diphenyloxy, and the like. At least one hydrogen atom of the alkoxy group can be substituted with the substituent described above for the phenyl group.

Other groups besides the groups described above are regarded as conventional meaning to those of ordinary skill in the art.

More particularly, according to an embodiment of the present invention, the heteroaromatic cycle-containing compound used for an organic light emitting diode may be a compound represented by Formula 2 below:

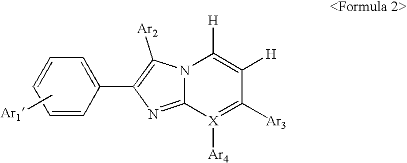

wherein Ar₁′ is a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group, and

X, Ar₂, Ar₃ and Ar₄ are the same as defined above.

According to an embodiment of the present invention, Ar₁′ of Formula 2 is any one of compounds below.

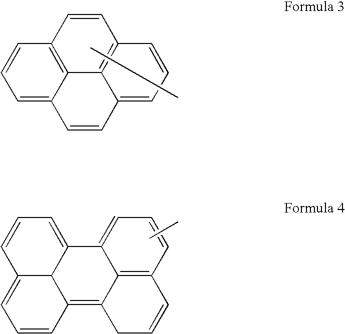

According to an embodiment of the present invention, Ar₂ is a hydrogen atom, methyl or phenyl.

According to an embodiment of the present invention, X is N, Ar₃ is hydrogen atom, and A₄ is a lone electron pair.

According to an embodiment of the present invention, X is C, Ar₃ and A₄ are hydrogen atoms or phenyl which is ortho-fused to the heteroaromatic cycle.

According to an embodiment of the present invention, the heteroaromatic cycle-containing compound for use in an organic light emitting diode may be compounds represented by Formulas 5 through 16 (which are also referred to as Compounds 1 through 12, respectively), but is not limited thereto.

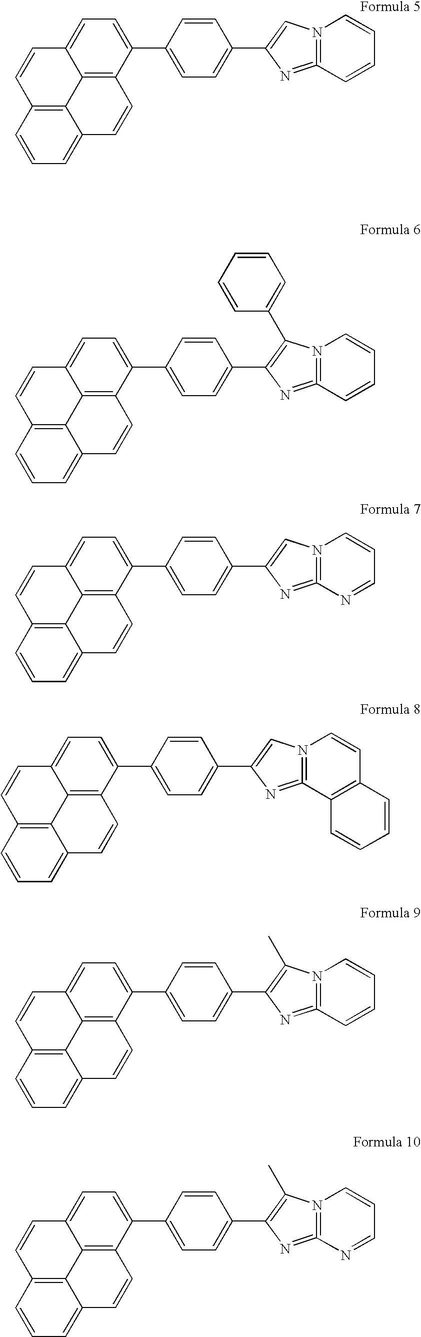

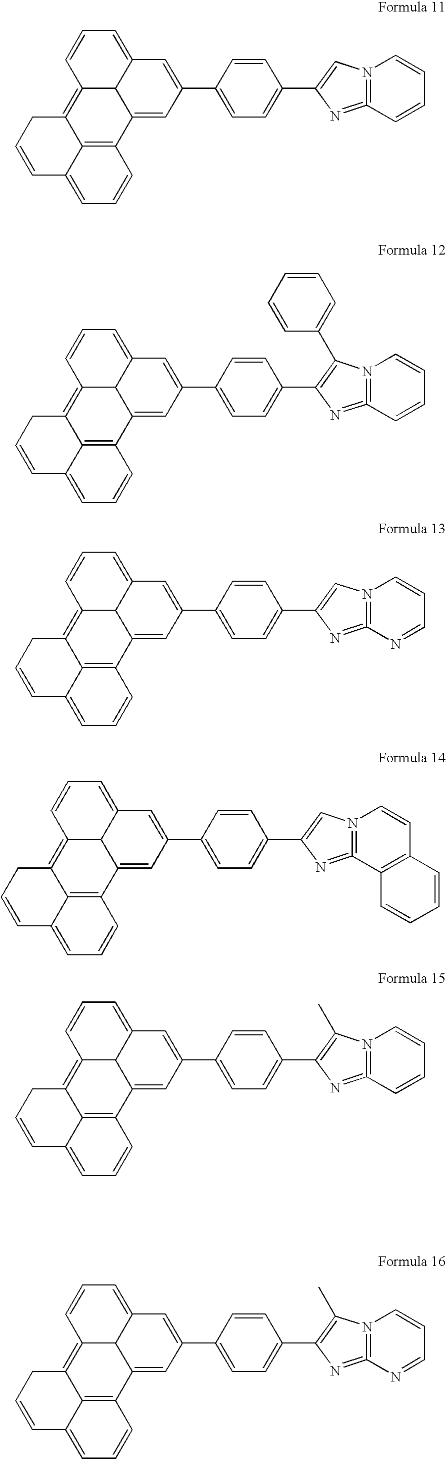

The position of the substituent of the heteroaromatic cycle-containing compound used for an organic light emitting diode, according to the present invention will now be described with reference to the following formula.

The inventors of the present invention found that among positions 2 through 7, when both hydrogens in positions 4 and 5 of the heteroaromatic cycle-containing compound shown in the formula below are unsubstituted, it effectively helps stacking between molecules, and significantly enhances electron transportation.

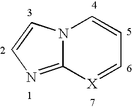

The heteroaromatic cycle-containing compound can be used as an electron transport layer material, an electron injection layer material and an emissive layer material. Using the compound, an organic light emitting diode having long life-time and high efficiency can be obtained.

The heteroaromatic cycle-containing compound used for an organic light emitting diode of Formula 1 may be prepared according to Reaction Scheme 1 below.

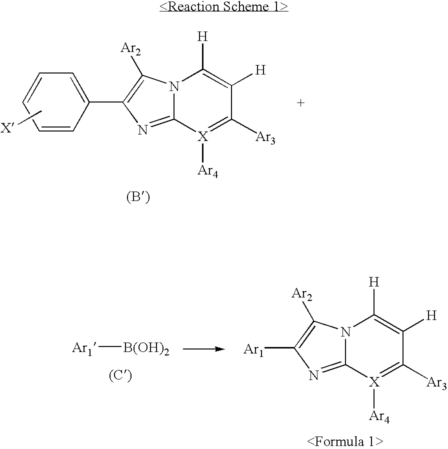

In Reaction Scheme 1, (B′) and (C′) are an imidazole derivative and a boronic acid derivative, respectively.

In Reaction Scheme 1, Ar₁′ and Ar₁ are each independently a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group, X, Ar₂, Ar₃ and Ar₄ are the same as defined above, and X′ is a halogen atom, for example, chloride, bromide, iodide, or the like.

The reaction is performed in the presence of tetrakis(triphenylphosphine)palladium (Pd(PPh₃)₄) and a base. The reaction is optimally performed in the reaction temperature range of 50-120° C.

Examples of the base may include potassium carbonate, sodium hydroxide, sodium hydrogen carbonate, and the like.

In Reaction Scheme 1, Ar₂ of the imidazole derivative (B′) can be introduced by a reaction of an imidazole derivative (D′) below and a boronic acid derivative (E′) below as in Reaction Scheme 2 below.

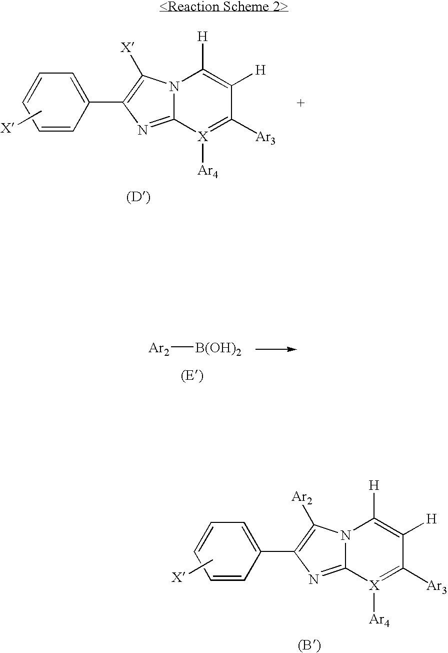

In Reaction Scheme 2, Ar₂, Ar₃ and Ar₄ are the same as defined above, Ar₃ and Ar₄ may be alternatively bound to each other to form a saturated or unsaturated carbon ring when X is C, Ar₄ refers to a lone electron pair when X is N, and X′ is a halogen atom, for example, chloride, bromide, iodide, or the like, each X′ being the same or different.

The reaction in Reaction Scheme 2 is performed in the presence of Pd(PPh₃)₄ and a base. The reaction is optimally performed in the reaction temperature range of 50-120° C.

In Reaction Scheme 2, the imidazole derivative (D′) can be produced by a reaction of an imidazole derivative (F′) below and N-halosuccinimide below as in Reaction Scheme 3 below.

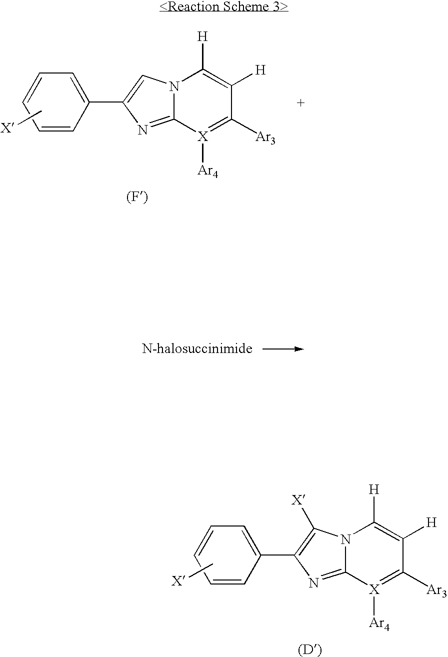

In Reaction Scheme 3, Ar₃ and Ar₄ are the same as defined above, Ar₃ and Ar₄ may be alternatively bound to each other to form a saturated or unsaturated carbon ring when X is C, Ar₄ refers to a lone electron pair when X is N, and X′ is a halogen atom, for example, chloride, bromide, iodide, or the like, each X′ being the same or different.

N-halosuccinimide is a reagent used in halogenation, for example, N-iodosuccinimide, N-bromosuccinimide, N-chlorosuccinimide, or the like.

In Reaction Scheme 3, the imidazole derivative (F′) can be produced by a reaction of α-halo ketone derivative (H′) and a heteroarylamine derivative (G′) as in Reaction Scheme 4 below.

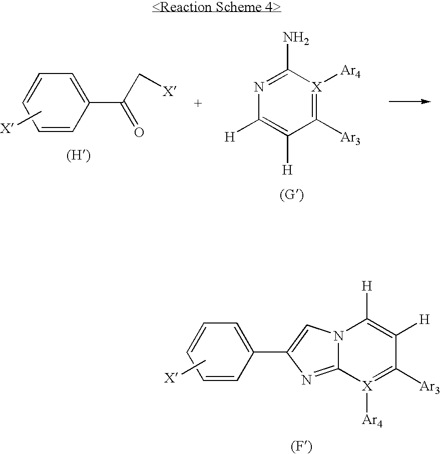

In Reaction Scheme 4, Ar₃ and Ar₄ are the same as defined above, Ar₃ and Ar₄ are alternatively bound to each other to form a saturated or unsaturated carbon ring when X is C, Ar₄ refers to a lone electron pair when X is N, and X′ is a halogen atom, each X′ being the same or different.

An organic light emitting diode according to an embodiment of the present invention includes a single-layered or multi-layered organic layer disposed between a first electrode and a second electrode, wherein the organic layer can comprise the heteroaromatic cycle-containing compound represented by Formula 1 as described above.

The organic light emitting diode has various structures. The organic layer formed between the first electrode and the second electrode may comprise at least one selected from the group consisting of a hole injection layer, a hole transport layer, an emissive layer, a hole blocking layer, an electron transport layer and an electron injection layer. The organic layer can comprise the heteroaromatic cycle-containing compound of Formula 1 as described above. For example, in the organic light emitting diode according to an embodiment of the present invention, the organic layer comprising the heteroaromatic cycle-containing compound of Formula 1 may be preferably an electron transport layer or an electron injection layer.

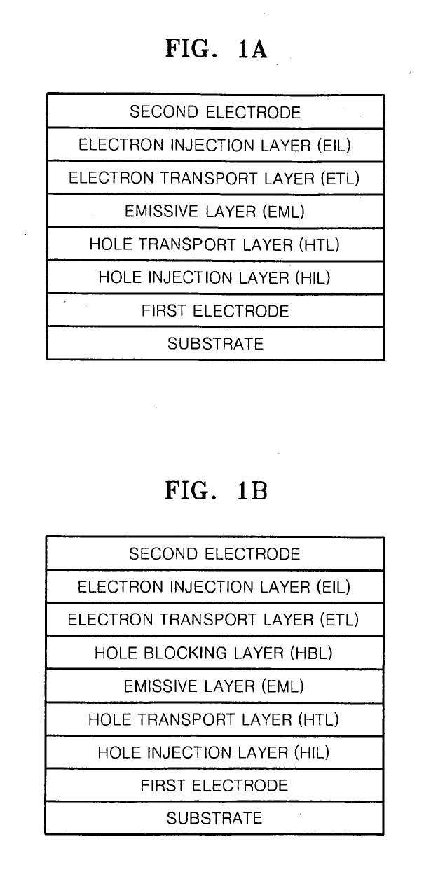

More particularly, FIGS. 1A and 1B are sectional views illustrating structures of organic light emitting diodes according to embodiments of the present invention.

The organic light emitting diode of FIG. 1A has a first electrode/hole injection layer/hole transport layer/emissive layer/electron transport layer/electron injection layer/second electrode structure. The organic light emitting diode of FIG. 1B has a first electrode/hole injection layer/hole transport layer/emissive layer/hole blocking layer/electron transport layer/electron injection layer/second electrode structure. Herein, the electron transport layer or the electron injection layer can comprise the heteroaromatic cycle-containing compound represented by Formula 1.

A method of manufacturing an organic light emitting diode having a multi-layered structure described above will now be described.

First, a first electrode, which can be an anode, is formed on a substrate using an anode material having a high work function by deposition or sputtering. The substrate, which can be any substrate that is used in conventional organic light emitting diodes, may be a glass substrate or a transparent plastic substrate that has excellent transparency and surface smoothness, can be easily treated, and is waterproof. The anode material can be a transparent and highly conductive material such as ITO, IZO, SnO₂, ZnO, or the like.

A hole injection layer material is vacuum-thermal-deposited or spin coated on the anode. Examples of the hole injection layer material may include, for example, a phthalocyanine compound, such as copper phthalocyanine (CuPc), disclosed in U.S. Pat. No. 4,356,429 which is incorporated herein by reference; a star-burst type amine derivative, such as TCTA, m-MTDATA, m-MTDAPB, disclosed in Advanced Material, 6, pp. 677 (1994) which is incorporated herein by reference; soluble and conductive polymer such as polyaniline/Dodecylbenzenesulfonic acid (PANI/DBSA); poly(3,4-ethylenedioxythiophene)/poly(4-styrenesulfonate) (PEDOT/PSS): polyaniline/camphor sulfonic acid (Pani/CSA); (polyaniline)/poly(4-styrenesulfonate) (PANI/PSS); or the like, but are not limited thereto.

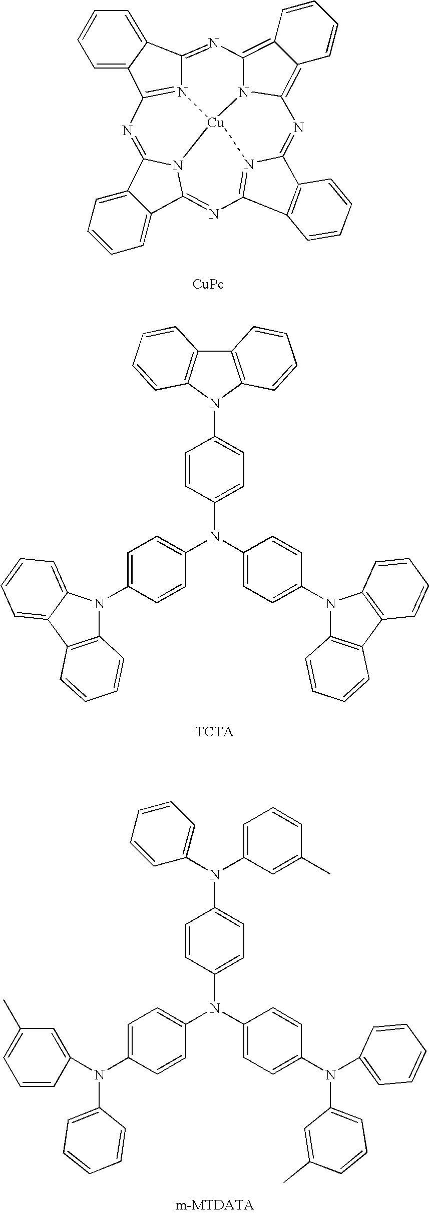

A hole transport layer material is vacuum-thermal-deposited or spin coated on the hole injection layer to form a hole transport layer. Examples of the hole transport layer material include 1,3,5-tricarbazolylbenzene, 4,4′-biscarbazolylbiphenyl, polyvinylcarbazole, m-biscarbazolylphenyl, 4,4′-biscarbazolyl-2,2′-dimethylbiphenyl, 4,4′,4″-tri(N-carbazolyl)triphenylamine, 1,3,5-tri(2-carbazolylphenyl)benzene, 1,3,5-tris(2-carbazolyl-5-methoxyphenyl)benzene, bis(4-carbazolylphenyl)silane, N,N′-bis(3-methylphenyl)-N,N′-diphenyl-[1,1-biphenyl]-4,4′diamine (TPD), N,N′-di(naphthalene-1-yl)-N,N′-diphenyl benzidine (α-NPD), N,N′-diphenyl-N,N′-bis(1-naphthyl)-(1,1′-biphenyl)-4,4′-diamine (NPB), poly(9,9-dioctylfluorene-co-N-(4-butylphenyl)diphenylamine (TFB), poly(9,9-dioctylfluorene-co-bis-(4-butylphenyl-bis-N,N-phenyl-1,4-phenylenediamin (PFB), and the like, but are not limited thereto.

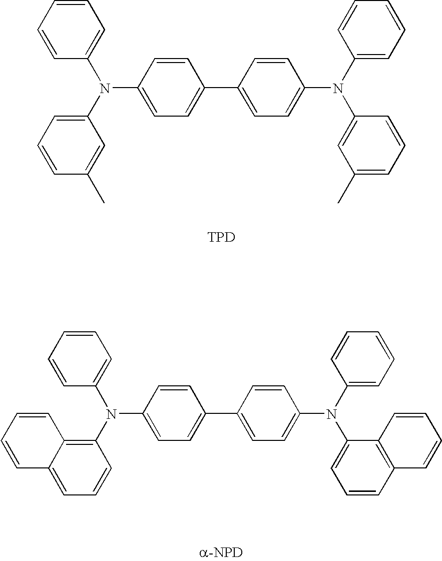

Next, an emissive layer is formed on the hole transport layer. An emissive layer material is not particularly limited, and 4,4′-biscarbazolylbiphenyl (CBP), TCB, TCTA, SDI-BH-18, SDI-BH-19, SDI-BH-22, SDI-BH-23, dmCBP, Liq, TPBI, Balq, BCP, or the like can be used as a host. As for a dopant, IDE102 and IDE105 available from Idemitsu Kosan Co., Ltd. as a fluorescent dopant, a well-known green phosphorescent dopant Ir(ppy)₃, a blue phosphorescent dopant (4,6-F2 ppy)₂Irpic, or the like can be co-deposited by vacuum-thermal-deposition.

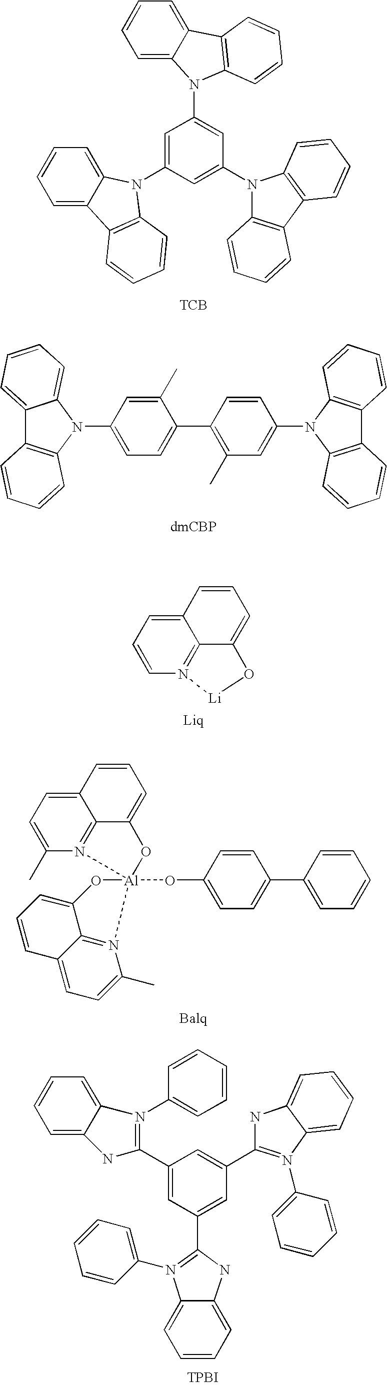

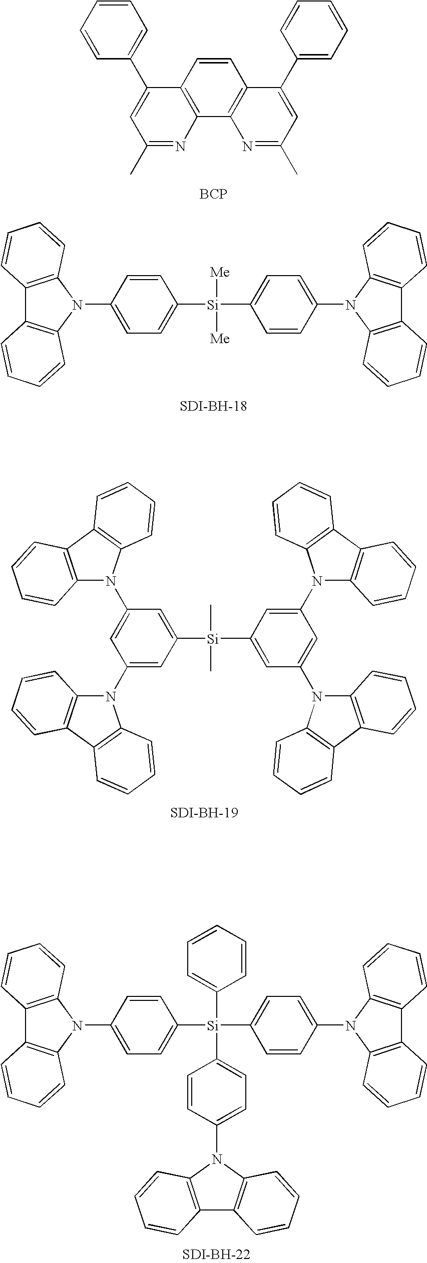

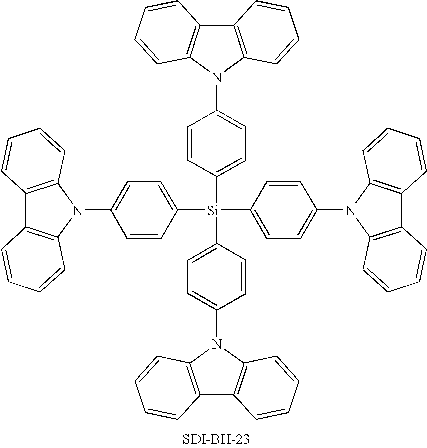

A doping concentration is not particularly limited, but conventionally in the range of 0.5-12 wt %.

An electron transport layer can be formed as a thin film on the emissive layer by vacuum deposition or spin coating.

When a phosphorescent dopant is used to form the emissive layer, a hole blocking material is additionally vacuum-thermal-deposited on the emissive layer to form a hole blocking layer, in order to prevent triplet excitons or holes from migrating into an electron transport layer. A hole blocking layer material used herein is not particularly limited, but has to provide the ability to transport electrons and have higher ionization potential than a light emitting compound. Examples of the hole blocking layer material include Balq, BCP, and the like.

An electron transport layer can be formed as a thin film on the hole blocking layer by vacuum deposition or spin coating. An electron transport layer material can be the heteroaromatic cycle-containing compound represented by Formula 1 and/or a known material such as Alq3, or the like.

In addition, an electron injection layer can be formed on the electron transport layer. Examples of an electron injection layer material include LiF, NaCl, CsF, Li₂O, BaO, and the like, but are not limited thereto.

Next, a cathode forming metal is vacuum-thermal-deposited on the electron injection layer to form a cathode. As a result, an organic light emitting diode is completed. The cathode forming metal can be Li, Mg, Al, Al—Li, Ca, Mg—In, Mg—Ag, or the like. In addition, in the structure of an electrode, a hole injection layer, a hole transport layer, an emissive layer, a hole blocking layer, an electron transport layer, an electron injection layer and a cathode, a transparent cathode made of ITO and IZO can be used as the cathode in order to obtain a single layer top-emitting device. An organic light emitting diode according to an embodiment of the present invention may further include an anode or a dual-layered intermediate layer.

Heteroaromatic cycle-containing compounds according to embodiments of the present invention expressed as Compound 1 through 4 will now be more fully described with reference to Synthesis Examples and Examples, but the present invention is not limited to the following examples.

### EXAMPLE

### Example 1

### Synthesis of Heteroaromatic Cycle-Containing Compound

Compound 1 represented by Formula 5 was synthesized according to Reaction Scheme 5 below.

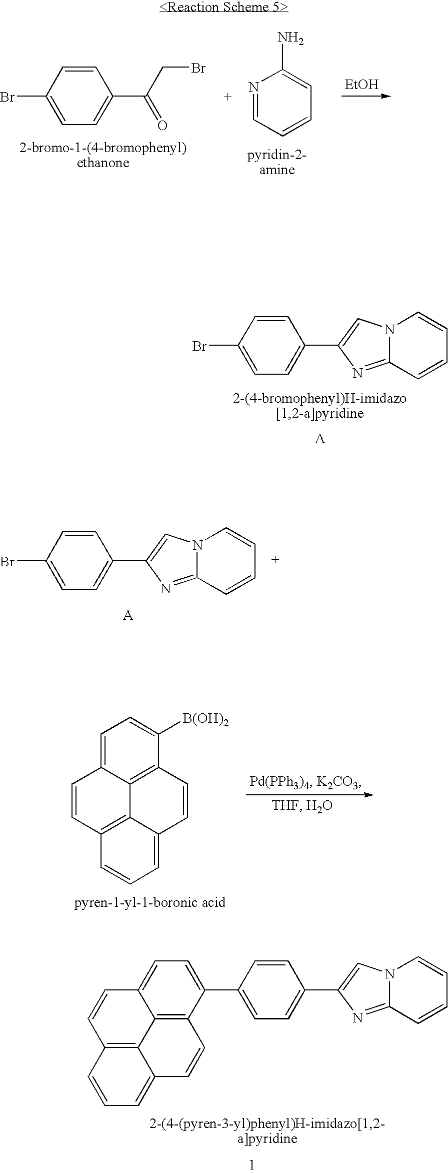

### Synthesis of Compound A (2-(4-bromophenyl)H-imidazo[1,2-a]pyridine)

10 g of 2-bromo-1-(4-bromophenyl)ethanone and 3.4 g of pyridine-2-amine were dissolved in 50 ml of ethanol, and then stirred at a reflux temperature for 18 hours. The resultant was cooled down to room temperature, and then the obtained white solid was filtered, washed using ethanol and ethylether to obtain 5 g of Compound A.

### Synthesis of Compound 1 (2-(4-(pyren-3-yl)phenyl)H-imidazo[1,2-a]pyridine)

5 g of Compound A, 4.5 g of pyren-1-yl-1-boronic acid, 0.46 g of tetrakis(triphenylphosphine)palladium, and 6 g of potassium carbonate were dissolved in 80 ml of tetrahydrofuran and 80 ml of water, and then stirred at a reflux temperature for 18 hours. The resultant was cooled down to room temperature, and then an organic layer was isolated and a water layer was extracted with 80 ml of dichloromethane. The obtained organic layer was dried with magnesium sulfide, and a solvent was evaporated to obtain a crude product. The crude product was purified with silica gel column chromatography to obtain 5.5 g of Compound 1.

¹H NMR (DMSO-d6, 400 MHz) δ (ppm) 8.56 (1H, d), 8.51 (1H, s), 8.36 (1H, d), 8.31 (1H, d), 8.28 (1H, d), 8.22˜8.18 (6H, m), 8.10˜8.05 (2H, m), 7.70 (2H, d), 7.61 (1H, d), 7.26 (1H, t), 6.91 (1H, t)

### Example 2

### Synthesis of Heteroaromatic Cycle-Containing Compound

Compound 2 represented by Formula 6 was synthesized according to Reaction Scheme 6 below.

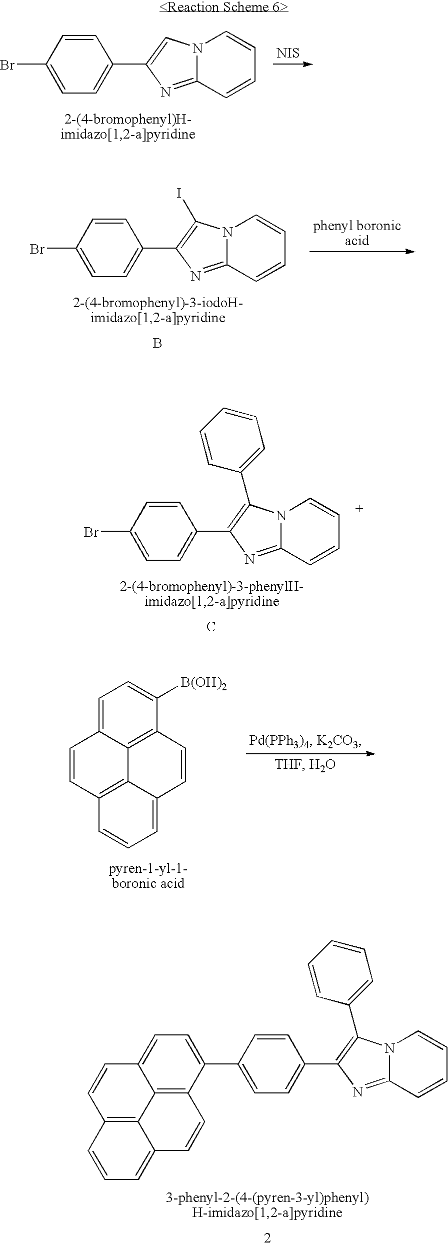

### Synthesis of Compound B (2-(4-bromophenyl)-3-iodoH-imidazo[1,2-a]pyridine)

5 g of Compound A (refer to Synthesis Example 1) and 4.12 g of N-iodosuccinimide were dissolved in an acetonitrile solvent, and then stirred at room temperature for 1 hour. Then, 100 ml of chloroform was added to the mixture, and the mixture was washed with 10% of an aqueous sodium hydride solution and then washed with an aqueous sodium thiosulfate saturated solution and water. The resultant was dried with anhydrous magnesium sulfate, and then a solvent was removed. Then, the obtained solid was washed with methanol and filtered to obtain 5.8 g of Compound B.

### Synthesis of Compound C (2-(4-bromophenyl)-3-phenylH-imidazo[1,2-a]pyridine)

5.8 g of Compound B, 1.8 g of phenylboronic acid, 335 mg of tetrakis(triphenylphosphine)palladium, and 10 g of potassium carbonate were dissolved in 80 ml of tetrahydrofuran and 80 ml of water, and then stirred at a reflux temperature for 18 hours. The mixture was cooled down to room temperature, and then an organic layer was isolated and a water layer was extracted with 80 ml of dichloromethane. The obtained organic layer was dried with magnesium sulfide, and a solvent was evaporated to obtain a crude product. The crude product was purified with silica gel column chromatography to obtain 2.9 g of Compound C.

### Synthesis of Compound 2 (3-phenyl-2-(4-(pyren-3-yl)Phenyl)H-imidazo[1,2-a]pyridine)

2.9 g of Compound C, 2.0 g of pyren-1-yl-1-boronic acid, 1.03 g of tetrakis(triphenylphosphine)palladium, and 13 g of potassium carbonate were dissolved in 40 ml of tetrahydrofuran and 40 ml of water, and then stirred at a reflux temperature for 18 hours. The mixture was cooled down to room temperature, and then an organic layer was isolated and a water layer was extracted with 40 ml of dichloromethane. The obtained organic layer was dried with magnesium sulfide, and a solvent was evaporated to obtain a crude product. The crude product was purified with silica gel column chromatography to obtain 3.0 g of Compound 2.

¹H NMR (DMSO-d6, 400 MHz) d (ppm) 8.24˜8.13 (4H, m), 8.07 (2H, s), 8.01˜7.97 (4H, m), 7.89 (2H, d), 7.73 (1H, d), 7.57 (7H, br s), 7.22 (1H, t), 6.74 (1H, t)

### Example 3

### Synthesis of Heteroaromatic Cycle-Containing Compound

Compound 3 represented by Formula 7 was synthesized according to Reaction Scheme 7 below.

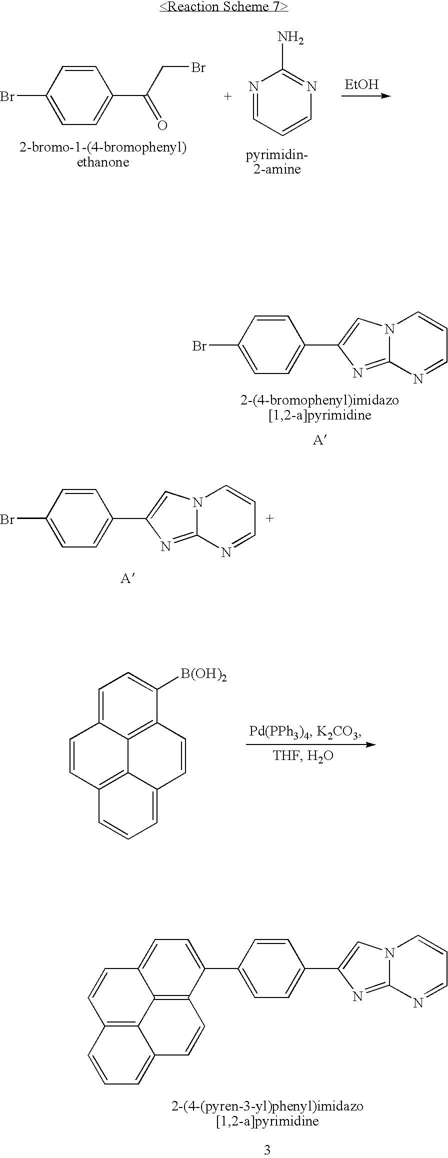

### Synthesis of Compound A′ (2-(4-bromophenyl)imidazo[1,2-a]pyrimidine)

10 g of 2-bromo-1-(4-bromophenyl)ethanone and 3.4 g of pyrimidine-2-amine were dissolved in 50 ml of ethanol, and then stirred at a reflux temperature for 18 hours. The mixture was cooled down to room temperature, and then the obtained white solid was filtered and washed with ethanol and ethylether to obtain 4.7 g of Compound A′.

### Synthesis of Compound 3 (2-(4-(pyren-3-yl)phenyl)imidazo[1,2-a]pyrimidine)

3.3 g of Compound A′, 3 g of pyren-1-yl-1-boronic acid, 0.7 g of tetrakis(triphenylphosphine)palladium, and 8 g of potassium carbonate were dissolved in 70 ml of tetrahydrofuran and 70 ml of water, and then stirred at a reflux temperature for 18 hours. The mixture was cooled down to room temperature, and then the solid product produced in an organic layer was filtered and washed with water and tetrahydrofuran to obtain 2.7 g of Compound 3.

¹H NMR (DMSO-d6, 400 MHz) d (ppm) 9.01 (1H, dd), 8.56 (1H, q), 8.51 (1H, s), 8.39 (1H, d), 8.32 (2H, q), 8.25˜8.19 (6H, m), 8.10 (2H, t), 7.75 (2H, d), 7.09 (1H, dd)

### Example 4

### Synthesis of Heteroaromatic Cycle-Containing Compound

Compound 4 represented by Formula 8 was synthesized according to Reaction Scheme 8 below.

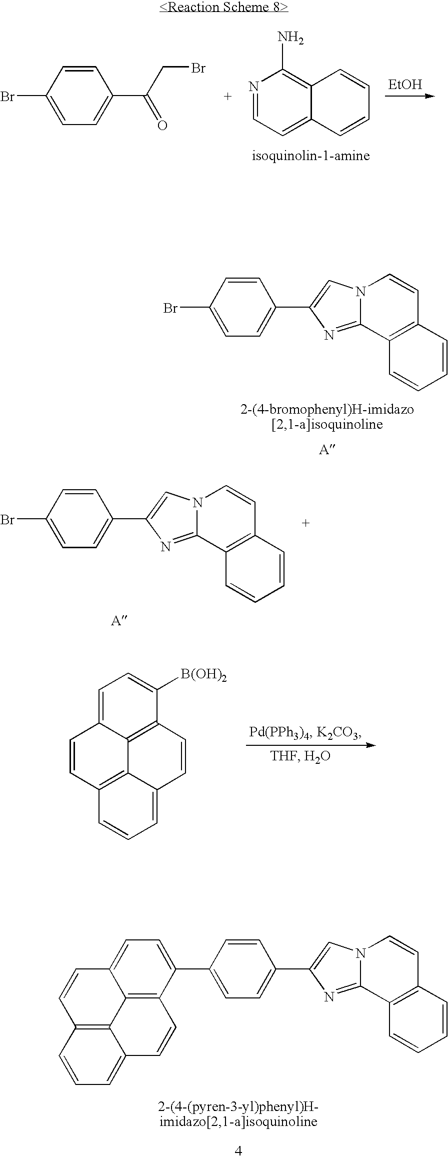

### Synthesis of Compound A″ (2-(4-bromophenyl)H-imidazo[2,1-a]isoquinoline)

7.7 g of 2-bromo-1-(4-bromo-phenyl)-ethanone and 4 g of isoquinoline-1-amine were dissolved in 100 ml of ethanol, and then stirred at a reflux temperature for 18 hours. The mixture was cooled down to room temperature, and then the obtained white solid was filtered and washed with ethanol and ethylether to obtain 6.7 g of Compound A″.

### Synthesis of Compound 4 (2-(4-(pyren-3-yl)Phenyl)H-imidazo[2,1-a]isoquinoline)

3.94 g of Compound A″, 3 g of pyren-1-yl-1-boronic acid, 0.7 g of tetrakis(triphenylphosphine)palladium, and 8 g of potassium carbonate were dissolved in 70 ml of tetrahydrofuran and 70 ml of water, and then stirred at a reflux temperature for 18 hours. The mixture was cooled down to room temperature, and then an organic layer was isolated and a water layer was extracted with 70 ml of dichloromethane. The obtained organic layer was dried with magnesium sulfide, and a solvent was evaporated to obtain a crude product. The crude product was purified with silica gel column chromatography to obtain 3.7 g of Compound 4.

¹H NMR (DMSO-d6, 400 MHz) d (ppm) 8.59 (1H, d), 8.56 (1H, s), 8.41 (1H, d), 8.40 (1H, d), 8.34 (1H, d), 8.32 (1H, d), 8.27˜8.19 (6H, m), 8.13˜8.10 (2H, m), 7.91 (1H, d), 7.75 (2H, d), 7.72˜7.66 (2H, m), 7.31 (1H, d)

A process of manufacturing an organic light emitting diode using Compounds 1 and 3 respectively synthesized in Examples 1 and 3, will now be described.

### Example 5

### ITO glass/m-MTDATA(750A)/αNPD(150A)/DSA(300A):TBPe(3%)/Compound 1(200A)/LiF(80A)/Al(3000A)

As an anode, a 15 Ω/cm² (1200 Å) Corning ITO glass substrate was cut to a size of 50 mm×50 mm×0.7 mm, washed with ultrasonic waves in isopropyl alcohol and pure water for 5 minutes each, and then cleaned with UV and ozone for 30 minutes. m-MTDATA was vacuum-deposited on the glass substrate to form a hole injection layer having a thickness of 750 Å. Then, α-NPD was vacuum-deposited on the hole injection layer to form a hole transport layer having a thickness of 150 Å. Next, distyrylanthracene (DSA) used as a host and 3% of tetra(t-butyl)perylene (TBPe) used as a dopant were vacuum-deposited on the hole transport layer to form an emissive layer having a thickness of 300 Å. Then, Compound 1 was vacuum-deposited on the emissive layer to form an electron transport layer having a thickness of 200 Å. 80 Å of LiF (electron injection layer) and 3000 Å (cathode) of Al were sequentially vacuum-deposited on the electron transport layer to form an LiF/Al electrode. Finally, an organic light emitting diode as illustrated in FIG. 1A was completed.

### Example 6

### ITO glass/m-MTDATA(750A)/α-NPD(150A)/DSA(300A):TBPe(3%)/Compound 3(200A)/LiF(80A)/Al(3000A)

As an anode, a 15 Ω/cm² (1200 Å) Corning ITO glass substrate was cut to a size of 50 mm×50 mm×0.7 mm, washed with ultrasonic waves in isopropyl alcohol and pure water for 5 minutes each, and then cleaned with UV and ozone for 30 minutes. m-MTDATA was vacuum-deposited on the glass substrate to form a hole injection layer having a thickness of 750 Å. Then, α-NPD was vacuum-deposited on the hole injection layer to form a hole transport layer having a thickness of 150 Å. Next, DSA used as a host and 3% of TBPe used as a dopant were vacuum-deposited on the hole transport layer to form an emissive layer having a thickness of 300 Å. Then, Compound 3 was vacuum-deposited on the emissive layer to form an electron transport layer having a thickness of 200 Å. 80 Å of LiF (electron injection layer) and 3000 Å of Al (cathode) were sequentially vacuum-deposited on the electron transport layer to form an LiF/Al electrode. Finally, an organic light emitting diode as illustrated in FIG. 1A was completed.

### Comparative Example

### ITO glass/m-MTDATA(750A)/α-NPD(150A)/DSA(300A):TBPe(3%)/Alq3(200A)/LiF(80A)/Al (3000A)

As an anode, a 15 Ω/cm² (1200 Å) Corning ITO glass substrate (manufactured by Corning Inc.) was cut to a size of 50 mm×50 mm×0.7 mm, washed with ultrasonic waves in isopropyl alcohol and pure water for 5 minutes each, and then cleaned with UV and ozone for 30 minutes. m-MTDATA was vacuum-deposited on the glass substrate to form a hole injection layer having a thickness of 750 Å. Then, α-NPD was vacuum-deposited on the hole injection layer to form a hole transport layer having a thickness of 150 Å. Next, DSA used as a host and 3% of TBPe used as a dopant were vacuum-deposited on the hole transport layer to form an emissive layer having a thickness of 300 Å. Then, Alq3 was vacuum-deposited on the emissive layer to form an electron transport layer having a thickness of 200 Å. 80 Å of LiF (electron injection layer) and 3000 Å of Al (cathode) were sequentially vacuum-deposited on the electron transport layer to form an LiF/Al electrode. Finally, an organic light emitting diode as illustrated in FIG. 1A was completed.

### Evaluation Example

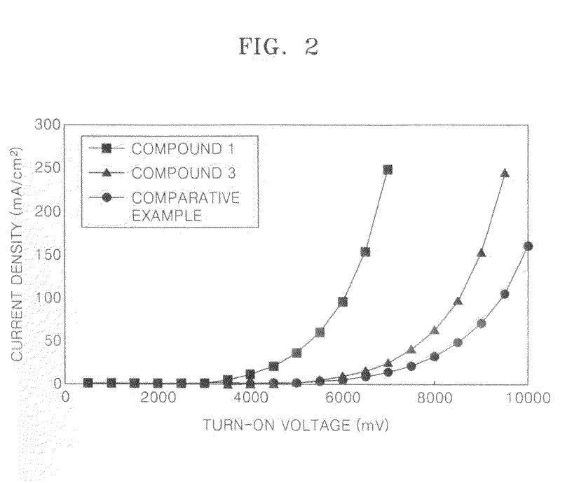

Evaluation results of current-voltage properties of the organic light emitting diodes of Example 5, Example 6 and Comparative Example are shown in Table 1 below, and FIG. 2 is graph showing the results. The current-voltage properties were evaluated using Keithley.

In addition, evaluation results of life-time properties of the organic light emitting diodes of Example 5, Example 6 and Comparative Example are shown in Table 1 below. The life-time properties were evaluated using Polaronix obtained from McScience Inc.

TABLE 1

| | | | Life-time |
|---|---|---|---|
| | Electron transport | | (luminance |
| | layer material | Turn-on voltage (at | half-life period at |
| | (200 Å) | 100 mA/cm²) | 100 mA/cm²) |
| Example 5 | Compound 1 | 6.0 V | 200 hours |
| Example 6 | Compound 3 | 8.4 V | 210 hours |
| Comparative | Alq3 | 9.5 V | 440 hours |
| Example | | | |

The heteroaromatic cycle-containing compound according to the present invention provides excellent electron transportation and has excellent stability, thus can be effectively used in a material for forming an organic layer. Using the compound, an organic light emitting diode that operates at low voltage and has long life-time can be obtained.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A heteroaromatic cycle-containing compound represented by Formula 1: 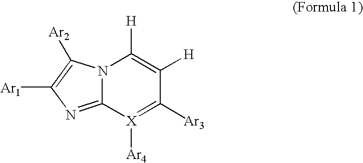
wherein X is N or C;
Ar₁ is a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group; and
Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring.

2. The heteroaromatic cycle-containing compound of claim 1, wherein the compound is represented by Formula 2 below: 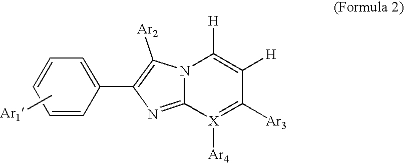
wherein X is N or C;
Ar₁′ is a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group; and
Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring.

3. The heteroaromatic cycle-containing compound of claim 2, wherein Ar₁′ is one of compounds represented by Formulas 3 and 4:

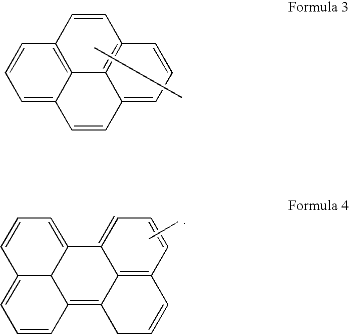

4. The heteroaromatic cycle-containing compound of claim 2, wherein Ar₂ is a hydrogen atom, methyl or phenyl.

5. The heteroaromatic cycle-containing compound of claim 2, wherein X is N, Ar₃ is hydrogen atom, and A₄ is a lone electron pair.

6. The heteroaromatic cycle-containing compound of claim 2, wherein X is C, Ar₃ and A₄ are hydrogen atoms or phenyl ortho-fused to the heteroaromatic cycle.

7. The heteroaromatic cycle-containing compound of claim 1, wherein the compound is one of Compounds represented by Formulas 5 through 16:

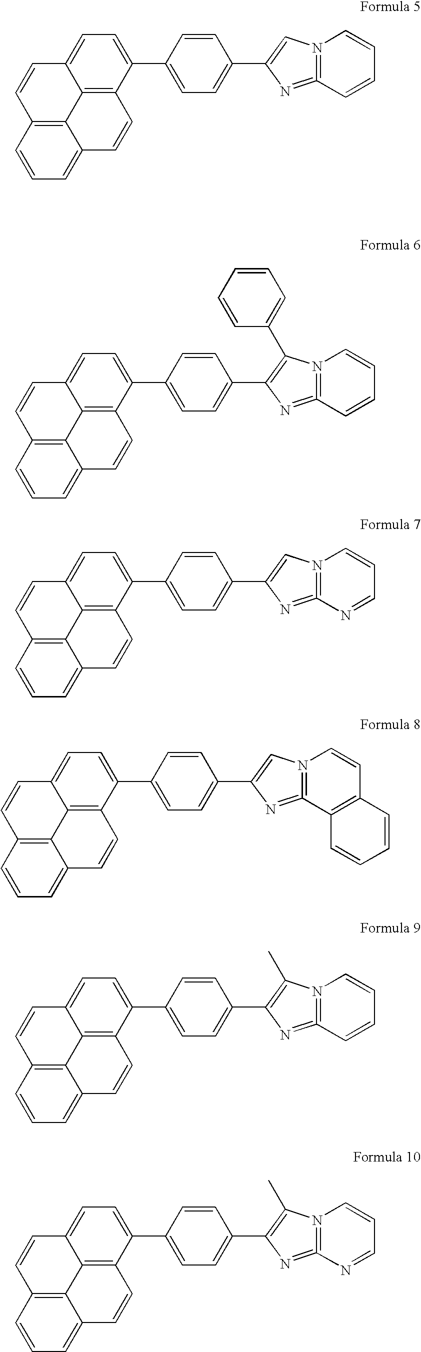

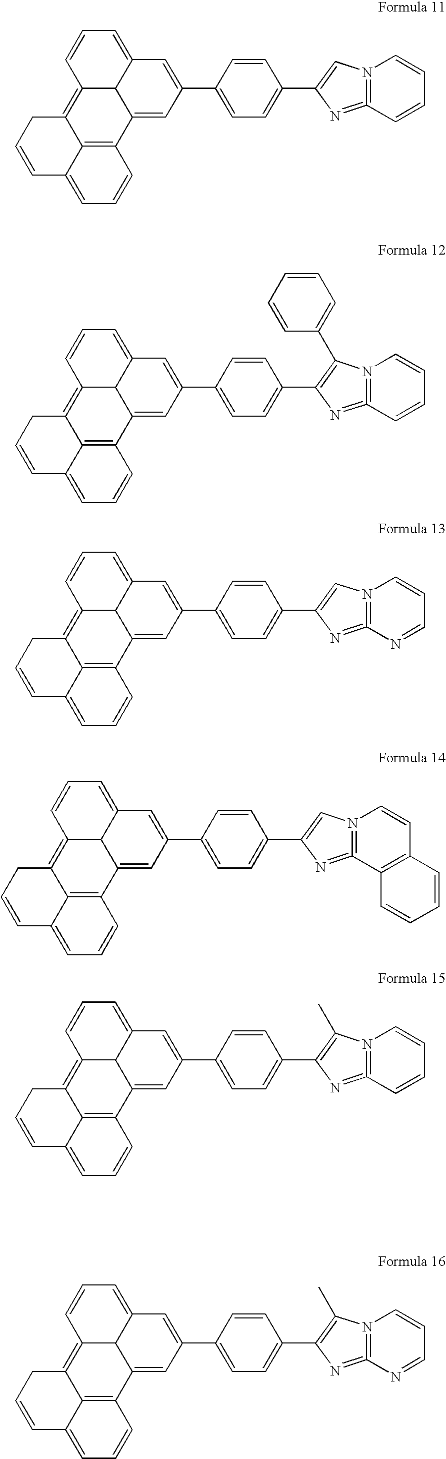

8. An organic light emitting device comprising at least one organic layer comprised of the heteroaromatic cycle-containing compound of claim 1.

9. A method of preparing a heteroaromatic cycle-containing compound represented by Formula 1, the method comprising:
reacting an imidazole derivative (B′) and a boronic acid derivative (C′): 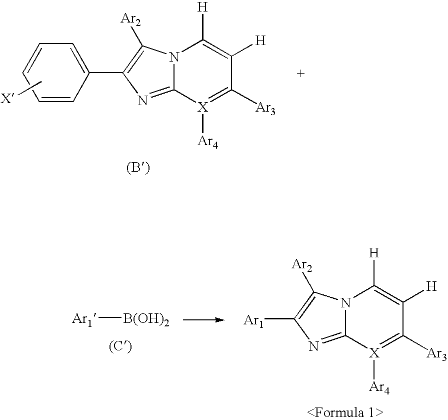
wherein X is N or C;
X′ is a halogen atom;
Ar₁′ and Ar₁ are each independently a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group; and
Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring.

10. The method of claim 9, wherein Ar₂ of the imidazole derivative (B′) is prepared by a reaction of an imidazole derivative (D′) and a boronic acid derivative (E′): 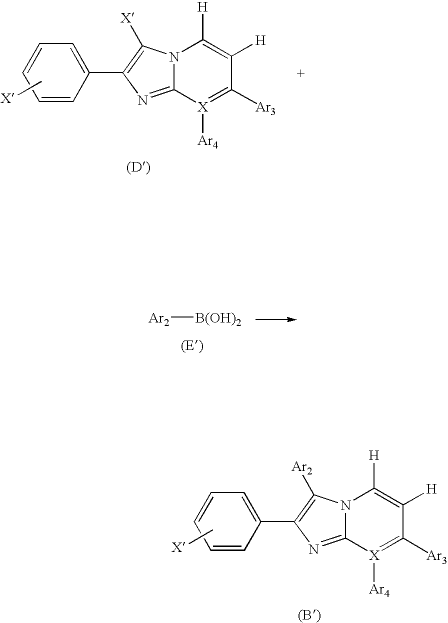
wherein X is N or C;
X′ is a halogen atom, wherein each X′ of D′ is the same halogen atom or different halogen atoms; and
Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring.

11. The method of claim 10, wherein the imidazole derivative (D′) is prepared by a reaction of an imidazole derivative (F′) and N-halosuccinimide: 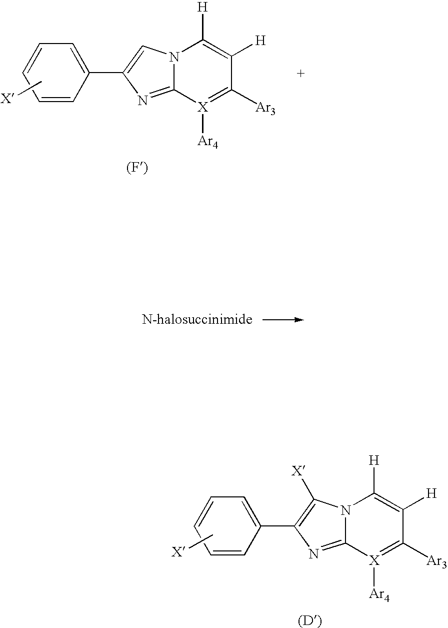
wherein X is N or C;
X′ is a halogen atom, wherein each X′ of D′ is the same halogen atom or different halogen atoms;
Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring; and
N-halosuccinimide is N-iodosuccinimide, N-bromosuccinimide, or N-chlorosuccinimide.

12. The method of claim 11, wherein the imidazole derivative (F′) is prepared by a reaction of α-halo ketone derivative (H′) and a heteroarylamine derivative (G′): 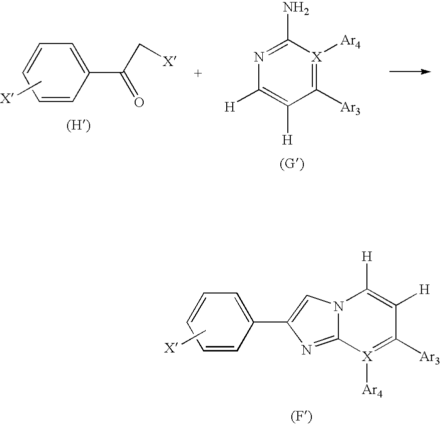
wherein X is N or C;
X′ is a halogen atom, wherein each X′ of H′ is the same halogen atom or different halogen atoms; and
Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring.

13. The method of claim 9, wherein the reaction is performed in the presence of Pd(PPh₃)₄ and a base, and the reaction temperature is in the range of 50 to 120° C.

14. The method of claim 10, wherein the reaction is performed in the presence of Pd(PPh₃)₄ and a base, and the reaction temperature is in the range of 50 to 120° C.

15. An organometallic complex prepared from the method of claim 9.

16. An organic light emitting diode comprising:
a first substrate;
a second substrate facing to the first substrate; and
at least one organic layer disposed between the first electrode and the second electrode, the at least one organic layer comprising an organic layer comprised of a heteroaromatic cycle-containing compound represented by Formula 1: 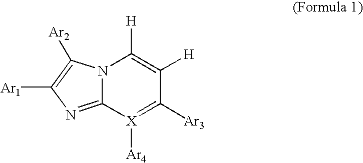
wherein X is N or C;
Ar₁ is a substituted or unsubstituted phenyl group, a substituted or unsubstituted C₆-C₂₀ aryl group, a substituted or unsubstituted C₇-C₂₀ arylalkyl group, a substituted or unsubstituted C₇-C₂₀ arylalkoxy group, a substituted or unsubstituted C₆-C₂₀ arylamino group, a substituted or unsubstituted C₆-C₂₀ heteroarylamino group, or a substituted or unsubstituted C₂-C₂₀ hetero ring group; and
Ar₂, Ar₃ and Ar₄ are each independently a hydrogen atom, a cyano group, a hydroxyl group, a nitro group, a halogen atom, a substituted or unsubstituted phenyl group, a substituted or unsubstituted C₁-C₂₀ alkyl group, a substituted or unsubstituted C₁-C₂₀ alkoxy group, a substituted or unsubstituted C₆-C₂₀ aryl group, a substituted or unsubstituted C₇-C₂₀ arylalkyl group, a substituted or unsubstituted C₂-C₂₀ alkylalkoxy group, a substituted or unsubstituted C₇-C₂₀ arylalkoxy group, a substituted or unsubstituted C₆-C₂₀ arylamino group, a substituted or unsubstituted C₁-C₂₀ alkylamino group, a substituted or unsubstituted C₆-C₂₀ heteroarylamino group, or a substituted or unsubstituted C₂-C₂₀ hetero ring group, wherein, when X is N, Ar₄ refers to a lone electron pair, and when X is C, Ar₃ and Ar₄ are alternatively bound to each other to form a saturated or unsaturated carbon ring.

17. The organic light emitting diode of claim 16, wherein the organic layer comprised of the heteroaromatic cycle-containing compound is an electron transport layer or an electron injection layer.

18. The organic light emitting diode of claim 16, wherein the organic layer comprised of the heteroaromatic cycle-containing compound is an emissive layer.

19. The organic light emitting diode of claim 16, wherein the at least one organic layer comprises at least one selected from the group consisting of a hole injection layer, a hole transport layer, an emissive layer, a hole blocking layer, an electron transport layer and an electron injection layer.

20. The organic light emitting diode of claim 14, wherein Ar₁′ is one of compounds represented by Formulas 3 and 4: 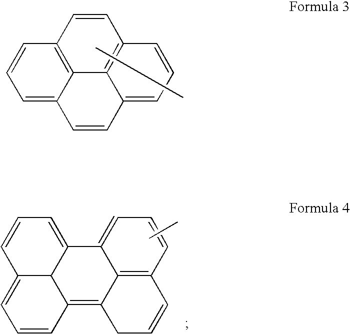
Ar₂ is a hydrogen atom, methyl or phenyl;
X is C or N;
when X is N, Ar₃ is hydrogen atom, and A₄ is a lone electron pair; and
when X is C, Ar₃ and A₄ are hydrogen atoms or phenyl ortho-fused to the heteroaromatic cycle.
